(19) United States
(12) Patent Application Publication
(10) Pub. No.: US 2024/0358865 A1
(43) Pub. Date: Oct. 31, 2024

(54) QUINOLINYL AND PRIDINYL CONTAINING COMPOUNDS TARGETING ON ALPHA-SYNUCLEIN FOR NEUROLOGICAL DISEASES

(71) Applicants: Washington University, St. Louis, MO (US); The Trustees of the University of Pennsylvania, Philadelphia, PA (US); University of Pittsburgh – Of the Commonwealth System of Higher Education, Pittsburgh, PA (US)

(72) Inventors: Zhude Tu, St. Louis, MO (US); Yanbo Yu, St. Louis, MO (US); Lin Qiu, St. Louis, MO (US); Paul Kotzbauer, St. Louis, MO (US); Robert Mach, Philadelphia, PA (US); Chester Mathis, Pittsburgh, PA (US)

(73) Assignees: Washington University, St. Louis, MO (US); The Trustees of the University of Pennsylvania, Philadelphia, PA (US); University of Pittsburgh – Of the Commonwealth System of Higher Education, Pittsburgh, PA (US)

(21) Appl. No.: 18/646,022

(22) Filed: Apr. 25, 2024

Related U.S. Application Data
(60) Provisional application No. 63/498,206, filed on Apr. 25, 2023.

(51) Int. Cl. A61K 51/04; C07B 59/00

(52) U.S. Cl. CPC A61K 51/0455; C07B 59/002; A61K 2123/00; C07B 2200/05

(57) ABSTRACT

The present disclosure is generally directed to quinolinyl and pyridinyl containing compounds targeting on alpha-synuclein for neurological diseases. These compounds, particularly radiolabeled versions of these compounds, can be particularly useful in PET scans targeting diseases such as Parkinson's disease (PD), Dementia with Lewy Bodies (DLB) and Multiple System Atrophy (MSA).

### CROSS REFERENCE TO RELATED APPLICATIONS

This application claims the benefit of U.S. Provisional Application No. 63/498,206, filed Apr. 25, 2023, the contents of which are incorporated by reference herein in their entirety.

### STATEMENT REGARDING FEDERALLY SPONSORED RESEARCH OR DEVELOPMENT

This invention was made with government support under NS110456 awarded by the National Institutes of Health. The government has certain rights in the invention.

### FIELD OF INVENTION

The present disclosure relates to compounds, particularly radiolabeled versions of these compounds, for use in targeting alpha-synuclein for neurological diseases. These can be particularly useful in PET scans targeting diseases such as Parkinson's disease (PD), Dementia with Lewy Bodies (DLB) and Multiple System Atrophy (MSA).

### BACKGROUND OF INVENTION

Neurodegenerative diseases such as Alzheimer's disease, Parkinson's disease (PD), Huntington's disease, amyotrophic lateral sclerosis, and prion diseases are debilitating diseases which affect cognition and/or muscle control. These diseases are a subset of protein misfolding diseases. Protein folding is an essential process for protein function in all organisms, and conditions that disrupt protein folding present a threat to cell viability. In some cases, the disease arises because a specific protein is no longer functional when adopting a misfolded state. In other diseases, the pathological state originates because misfolding occurs concomitantly with aggregation, and the underlying aggregates are detrimental.

Even though neurodegenerative diseases such as Alzheimer's and Parkinson's are caused by different proteins, both involve the accumulation of insoluble fibrous protein deposits, called amyloids. For example, Parkinson's Disease (PD), Dementia with Lewy Bodies (DLB), and multiple system atrophy (MSA), which are collectively referred to as “synucleinopathies,” have been linked to the accumulation of aggregated forms of the α-synuclein protein in neurons in the brain.

α-Synuclein is a presynaptic terminal protein that consists of 140-amino acid protein that plays an important function in the central nervous system including synaptic vesicle recycling and synthesis, vesicular storage, and neurotransmitter release. It is specifically upregulated in a discrete population of presynaptic terminals of the brain during acquisition-related synaptic rearrangement. α-Synuclein naturally exists in a highly soluble, unfolded state. Evidence suggests that filamentous aggregates of α-synuclein accumulate at the pre-synaptic membrane and trigger synapse dysfunction and neuronal cell death in synucleinopathies, and may be the cause of Parkinson's and DLB. α-Synuclein aggregation has been identified by antibody-immunohistological studies as the major component of Lewy bodies, which are microscopic protein deposits in deteriorating nerve cells. Accumulation of misfolded, fibrillar α-synuclein in Lewy bodies (LB) and Lewy neurites (LN) is considered a hallmark of PD.

In vivo imaging of α-synuclein aggregations with a clinically suitable positron emission tomography (PET) radioligand could provide important information in early diagnosis of PD, disease progression, and assessment of the efficacy of disease-modifying therapies. There remains a need for compounds, particularly radiolabeled compounds, that have high potency and high selectivity for alpha-synuclein binding that can be used for this purpose.

### SUMMARY OF INVENTION

The present disclosure provides a compound having a structure of any one of Formulae 1, 2, 3, 4, or 5, or a salt thereof:

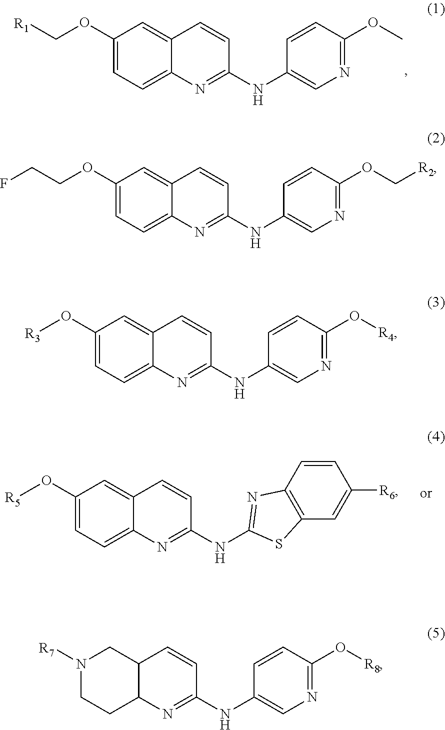

wherein:
R₁ is aryl, heteroaryl, halo-substituted C₃ alkyl, disubstituted halo-C₁-C₃ alkyl, trisubstituted halo-C₁-C₃ alkyl, cycloalkyl, alkylcycloalkyl, heterocycloalkyl, or alkylheterocycloalkyl;
R₂ is substituted C₁-C₅ alkyl or unsubstituted C₁-C₅ alkyl;
R₃, R₅, and R₈ are independently aryl, heteroaryl, alkaryl, alkheteroaryl, substituted C₁-C₆ alkyl, unsubstituted C₁-C₆ alkyl, substituted C₁-C₆ alkenyl, unsubstituted C₁-C₆ alkenyl, cycloalkyl, alkylcycloalkyl, heterocycloalkyl, or alkylheterocycloalkyl;
R₄ and R₆ are independently substituted C₁-C₆ alkyl, unsubstituted C₁-C₆ alkyl, substituted C₁-C₆ alkenyl, or unsubstituted C₁-C₆ alkenyl;
R₇ is hydrogen, C₁-C₆ alkyl, —OH, —OR₉, —NR₁₀R₁₁; and
R₉, R₁₀, and R₁₁ are independently substituted C₁-C₆ alkyl or unsubstituted C₁-C₆ alkyl.

The present disclosure is further directed to a pharmaceutical composition comprising a radiolabeled compound described herein and at least one excipient.

Another aspect of the invention is a method of diagnosing or monitoring a synucleinopathy in a human subject comprising administering the pharmaceutical composition disclosed herein to a human subject; and imaging the subject's brain by positron emission tomography.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1A-1B depict PET characterization of [¹⁸F]TZ83-41 and [¹⁸F]TZ80-156 in the macaque brain. FIG. 1A depicts time-activity curves for a microPET study in the brain, SUV: standardized uptake value. FIG. 1B depicts representative PET images of two radiotracers in the macaque brain, weighted average of 0-120 min. FIG. 1C depicts representative PET images of two radiotracers in the macaque brain for individual time points.

FIG. 2A-2B depict data for monkey plasma with ˜10 mCi radiotracer injection in monkey plasma and time points 5 min, 15 min, 30 min, 60 min, and 90 min, in vitro blood, dose. FIG. 2B depict the original radioactive curves.

FIG. 3A depicts [¹²⁵I]TZ61-84 binding in Banner BD, AD, and control tissues.

FIG. 3B depicts K_{d} and Bₘₐₓ value of [¹²⁵I]TZ61-84 in Banner BD tissue.

FIG. 3C depicts PET studies of [¹¹C]TZ61-84 in the brains of nonhuman primates.

FIG. 4A depicts brain uptake curves of [¹⁸F]TZ80-156 and [¹⁸F]TZ83-41 in nonhuman primates.

FIG. 4B depicts a PET brain study of α-Syn radiotracers.

FIG. 5A depicts the alpha-synuclein fibrils binding potency of new compounds determined using triatiated [³H]BF2846, a reported potent and non-selective alpha-synuclein ligand.

FIG. 5B depicts the binding potency for Abeta determined using [³H]PIB for these new compounds.

FIG. 6A depicts percent of [³H]—BF2846 specific binding.

FIG. 6B depicts percent of [³H]PiB specific binding.

FIGS. 7A-7C depict saturation binding assay results with 50 nM Banner PD-amplified fibrils and blocked with 0.3 μM cold TZ-80-156. FIG. 7A depicts global fit of total and non-specific binding. FIG. 7B depicts fit of specific binding only. FIG. 7C depicts non-specific binding.

FIGS. 8A-8C depict homologous competition binding assays.

### DETAILED DESCRIPTION OF INVENTION

As used in this application, including the appended claims, the singular forms “a,” “an,” and “the” include plural references unless the content clearly dictates otherwise, and are used interchangeably with “at least one” and “one or more.”

The invention will be further described in the following examples, which do not limit the scope of the invention described in the claims.

The aggregation of misfolded α-synuclein protein is a pathological hallmark of Parkinson's disease (PD), Dementia with Lewy Bodies (DLB) and Multiple System Atrophy (MSA). In vivo imaging of α-synuclein aggregations with a clinically suitable positron emission tomography (PET) radioligand could provide important information in early diagnosis of PD, disease progression, and assessment of the efficacy of disease-modifying therapies. Herein, it is reported that a series of compounds containing both of a quinolinyl ring and a pyridinyl aromatic ring have high potency and high selectivity for alpha-synuclein binding based on in vitro radioactive competitive binding studies. Carbon-11 or F-18 PET isotope can be easily introduced to these molecules to make C-11 or F-18 radiotracers for PET imaging quantification of the alpha-synuclein accumulation in the brain. To test the feasibility of measuring the alpha-synuclein in the brain in living animal, two lead ¹⁸F-labeled radioligands, [¹⁸F]TZ83-41 and [¹⁸F]TZ80-156, were successfully radiosynthesized, and initial nonhuman primate brain imaging and metabolite analysis of nonhuman plasma samples collected post injection of the radiotracer were performed. The data showed that [¹⁸F]TZ83-41, a representative for this series of compounds, had high brain uptake, suitable brain washout pharmacokinetics, and importantly, no lipophilic radiometabolite to enter into the brain and to cofound the PET measurement. Together, this series of compounds having high potency and selectivity for alpha-synuclein and have great potential to be therapeutic drugs targeting on alpha-synuclein. Further radiosynthesis and evaluation of their C-11 and F-18 radiotracers will lead to identifying PET imaging contrast agents for measurement of alpha-synuclein aggregation in the brain, which has great value in early diagnosis, prediction, monitoring the progress of PD and related diseases, and validating the efficacy of drugs targeting on alpha-synuclein.

### Compounds

One aspect of the present disclosure is a compound having a structure of any one of Formulae 1, 2, 3, 4, or 5, or a salt thereof:

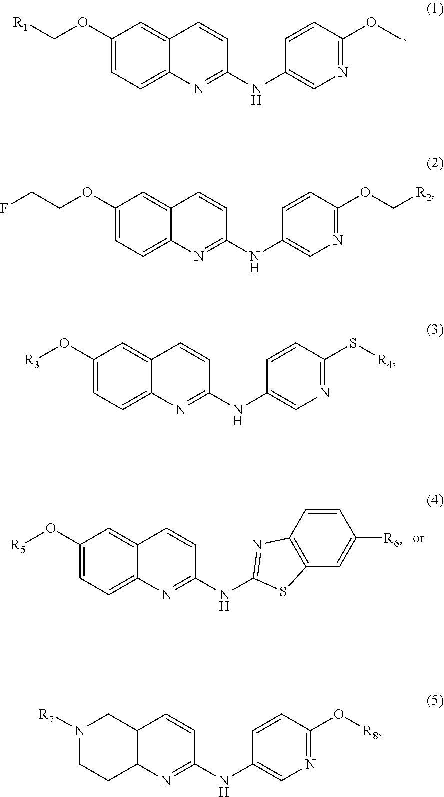

wherein:
R₁ is aryl, heteroaryl, halo-substituted C₃ alkyl, disubstituted halo-C₁-C₃ alkyl, trisubstituted halo-C₁-C₃ alkyl, cycloalkyl, alkylcycloalkyl, heterocycloalkyl, or alkylheterocycloalkyl;
R₂ is substituted C₁-C₅ alkyl or unsubstituted C₁-C₅ alkyl;
R₃, R₅, and R₅ are independently aryl, heteroaryl, alkaryl, alkheteroaryl, substituted C₁-C₆ alkyl, unsubstituted C₁-C₆ alkyl, substituted C₁-C₆ alkenyl, unsubstituted C₁-C₆ alkenyl, cycloalkyl, alkylcycloalkyl, heterocycloalkyl, or alkylheterocycloalkyl;
R₄ and R₆ are independently substituted C₁-C₆ alkyl, unsubstituted C₁-C₆ alkyl, substituted C₁-C₆ alkenyl, or unsubstituted C₁-C₆ alkenyl;
R₇ is hydrogen, C₁-C₆ alkyl, —OH, —OR₉, —NR₁₀R₁₁; and
R₉, R₁₀, and R₁₁ are independently substituted C₁-C₆ alkyl or unsubstituted C₁-C₆ alkyl.

The compound can have the structure of Formula 1, wherein R₁ is —CH₂— piperidyl, —CHF₂, —CF₃, pyridyl, or —CH═CH—CH₂F.

The compound can have the structure of Formula 2A

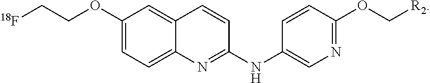

In various embodiments, R₂ can be methyl or ethyl.

The compound can have the structure of Formula 3, wherein R₄ is C₁-C₃ alkyl. In various embodiments, R₄ can be methyl.

The compound can have the structure of Formula 4, wherein R₆ is —OH or —N(CH₃)₂. In various embodiments, R₅ is substituted C₁-C₃ alkyl or unsubstituted C₁-C₃ alkyl. In various embodiments, R₅ is halo-substituted C₁-C₃ alkyl. In various embodiments, R₅ is unsubstituted C₁-C₃ alkyl.

The compound can have the structure of Formula 5, wherein R₈ is substituted C₁-C₆ alkyl or unsubstituted C₁-C₆ alkyl. In various embodiments, R₈ is substituted C₁-C₃ alkyl or unsubstituted C₁-C₃ alkyl. In various embodiments, R₈ is methyl. In various embodiments, R₇ is alkheteroaryl. In various embodiments, R₇ is —CH₂-pyridyl.

The compound can have the structure

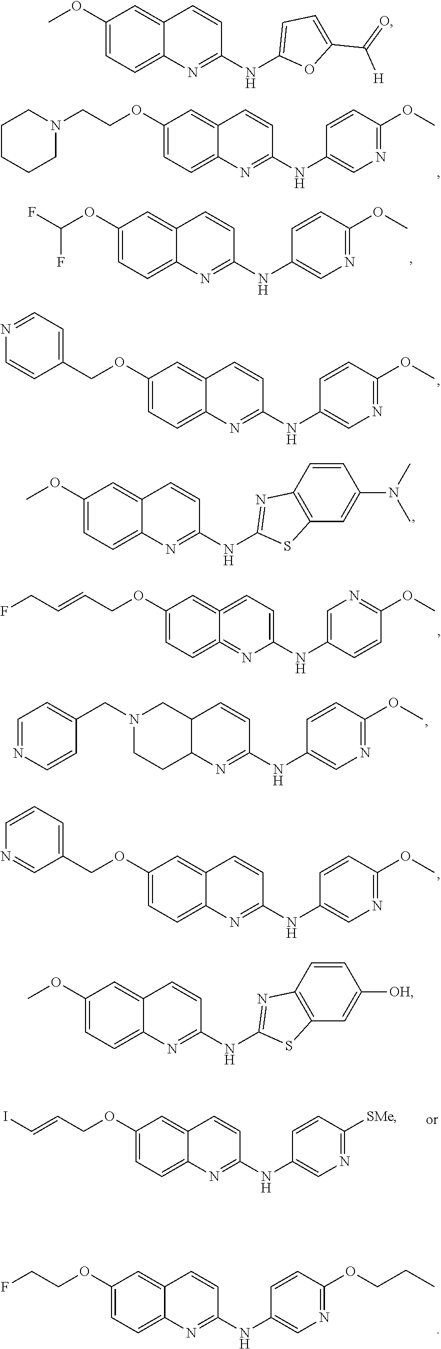

For any of the compounds disclosed herein, one or more atoms of the compound can be radiolabeled with a synthetic radioactive isotope. The synthetic radioactive isotope can be carbon-11, nitrogen-13, oxygen-15, fluorine-18, bromine-76, iodine-123, or iodine-125. In various embodiments, the synthetic radioactive isotope is fluorine-18 or carbon-11. The compound can have the structure

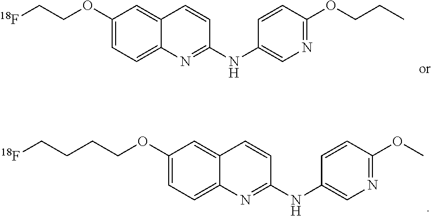 [SMILES: CCCOc1ccc(Nc2ccc3cc(OCC[18F])ccc3n2)cn1 ; COc1ccc(Nc2ccc3cc(OCCCC[18F])ccc3n2)cn1]

### Pharmaceutical Compositions or Formulations

As noted, the present disclosure relates to pharmaceutical compositions comprising a therapeutically effective amount of at least one of the compounds as described herein (e.g., a compound of any one of Formulae 1, 2, 3, 4, or 5, or a salt thereof).

Another aspect of the disclosure is a pharmaceutical composition comprising a pharmaceutically acceptable excipient and the compound of any one of Formulae 1, 2, 3, 4, or 5, or a salt thereof, described herein.

The pharmaceutical composition can also comprise a radiolabeled compound of the structure of any one of Formulae 1, 2, 3, 4, or 5, or a salt thereof, and at least one excipient. The composition can comprise from about 0.001 mg to about 10 g of the compound and at least about 10 wt. %, at least about 25 wt. %, at least about 50 wt. %, at least about 75 wt. %, at least about 90 wt. %, or at least about 95 wt. % of the compound in the pharmaceutical composition is radiolabeled.

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers. Pharmaceutically acceptable excipients for use in the compositions of the present invention are selected based upon a number of factors including the particular compound used, and its concentration, stability and intended bioavailability; the subject, its age, size and general condition; and the route of administration.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. Routes of administration include, but are not limited to, oral, parenteral (e.g., intravenous, intra-arterial, subcutaneous, rectal, subcutaneous, intramuscular, intraorbital, intracapsular, intraspinal, intraperitoneal, or intrasternal), topical (nasal, transdermal, intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual and intestinal administration. For example, the agents of use with the current disclosure can be formulated by known methods for administration to a subject using several routes including: parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

The pharmaceutical compositions can be formulated, for example, for oral administration. The pharmaceutical compositions can be formulated as tablets, dispersible powders, pills, capsules, gel-caps, granules, solutions, suspensions, emulsions, syrups, elixirs, troches, lozenges, or any other dosage form that can be administered orally. Pharmaceutical compositions can include one or more pharmaceutically acceptable excipients. Suitable excipients for solid dosage forms include sugars, starches, and other conventional substances including lactose, talc, sucrose, gelatin, carboxymethylcellulose, agar, mannitol, sorbitol, calcium phosphate, calcium carbonate, sodium carbonate, kaolin, alginic acid, acacia, corn starch, potato starch, sodium saccharin, magnesium carbonate, microcrystalline cellulose, colloidal silicon dioxide, croscarmellose sodium, talc, magnesium stearate, and stearic acid. Further, such solid dosage forms can be uncoated or can be coated to delay disintegration and absorption.

The pharmaceutical compositions can also be formulated for parenteral administration, e.g., formulated for injection via intravenous, intra-arterial, subcutaneous, rectal, subcutaneous, intramuscular, intraorbital, intracapsular, intraspinal, intraperitoneal, or intrasternal routes. Dosage forms suitable for parenteral administration include solutions, suspensions, dispersions, emulsions or any other dosage form that can be administered parenterally.

Pharmaceutically acceptable excipients are identified, for example, in The Handbook of Pharmaceutical Excipients, (American Pharmaceutical Association, Washington, D.C., and The Pharmaceutical Society of Great Britain, London, England, 1968). Additional excipients can be included in the pharmaceutical compositions of the invention for a variety of purposes. These excipients can impart properties which enhance retention of the compound at the site of administration, protect the stability of the composition, control the pH, facilitate processing of the compound into pharmaceutical compositions, and so on. Other excipients include, for example, fillers or diluents, surface active, wetting or emulsifying agents, preservatives, agents for adjusting pH or buffering agents, thickeners, colorants, dyes, flow aids, non-volatile silicones, adhesives, bulking agents, flavorings, sweeteners, adsorbents, binders, disintegrating agents, lubricants, coating agents, and antioxidants.

Compound described herein can be prepared as a salt. “Salt” as used herein refers to pharmaceutically acceptable salts of the compounds described herein which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66:1-19 (1977). Examples of pharmaceutically acceptable salts include, but are not limited to, nontoxic acid addition salts which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

In other embodiments, the compounds may be prepared as “prodrugs” in a pharmaceutically acceptable composition/formulation. As used herein, the term “prodrug” refers to a derivative of a compound that can hydrolyze, oxidize, or otherwise react under biological conditions (in vitro or in vivo) to provide a compound as described herein. Prodrugs may only become active upon some reaction under biological conditions, but they may have activity in their unreacted forms. Examples of prodrug moieties include substituted and unsubstituted, branch or unbranched lower alkyl ester moieties, (e.g., propionic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Prodrugs and their uses are well known in the art (see, e.g., Berge, et al. 1977 J. Pharm. Sci. 66:1-19). Prodrugs can typically be prepared using well-known methods, such as those described in Burger's Medicinal Chemistry and Drug Discovery (1995, Manfred E. Wolff ed., 5th ed. 172-178, 931-932).

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Methods

A further aspect of the invention is a method of diagnosing or monitoring a synucleinopathy in a human subject comprising administering a pharmaceutical composition described herein to a human subject; and imaging the subject's brain by positron emission tomography.

The synucleinopathy can correlate to a condition comprising Parkinson's Disease (PD), dementia with Lewy Bodies (DLB), or multiple system atrophy (MSA).

After radiolabeling with C-11 or F-18, I-125, I-123, H-3, and Br-76, compounds with high binding affinity and selectivity for aggregated α-synuclein can be used as radiotracers to measure the level and distribution of aggregated α-synuclein in patients with PD and related diseases such as DLB and MSA. Determining the level and distribution of aggregated α-synuclein could be useful for early diagnosis and for monitoring response to therapy in these diseases. They could also potentially be useful for treatment of PD and related diseases and monitor the therapeutic efficacy.

These compounds also could be a potential therapeutic drugs for treating PD and related diseases by targeting alpha-synuclein protein.

Generally, the present invention is directed to compounds that are useful α-synuclein ligands. The compounds possess sufficient binding affinity to α-synuclein fibrils. Also, various compounds of the present invention are highly selective ligands for α-synuclein as compared to other fibrils such as AB-fibrils and tau fibrils. As a result, radiolabeled analogs of the compounds described herein are useful for certain diagnostic methods for synucleinopathies such as PD.

The present invention is also directed to the α-synuclein ligands that are radiolabeled with radionuclides such as carbon-11 and/or fluorine-18 to serve as imaging agents (e.g., positron emission tomography (PET) imaging agents) for quantifying α-synuclein protein aggregation in the brain. The in vivo quantification of α-synuclein protein aggregation in patients is useful not only for diagnosing synucleinopathies such as PD, but also for monitoring disease progression.

As noted, fibrillar α-synuclein imaging is a highly useful marker for disease progression. Thus, an α-synuclein imaging agent provides for accurate enrollment of early stage PD patients into trials of therapeutic interventions targeting disease progression. If progressive accumulation of α-synuclein within individual regions or across multiple brain regions correlates with disease progression, particularly in early and intermediate disease stages, an α-synuclein imaging agent could also greatly improve evaluation of therapeutic efficacy for candidate disease-modifying interventions.

As noted, the compounds of the present invention possess binding affinity to α-synuclein which is useful for certain diagnostic and monitoring methods for synucleinopathies such as PD. One diagnostic method that is suitable for use with the α-synuclein ligands of the present invention is positron emission tomography (PET). PET is known in the art of nuclear medicine imaging as a non-invasive imaging modality that can provide functional information of a living subject at the molecular and cellular level. PET utilizes biologically active molecules in micromolar or nanomolar concentrations that have been labeled with short-lived positron emitting isotopes. The physical characteristics of the isotopes and the molecular specificity of labeled molecules, combined with the high detection efficacy of modern PET scanners provides a sensitivity for in vivo measurements of indicator concentrations that is several orders of magnitude higher than with other imaging techniques.

In order to make measurements with PET, a biologically active tracer molecule labeled with a positron-emitting isotope is administered to a subject, for example, intravenously, orally, or by inhalation. The subject is then scanned, and axial tomographic slices of regional cerebral tracer accumulation are obtained. This tracer accumulation can be related to cerebral metabolism, blood flow, or binding site concentrations by appropriate mathematical models. Thus, by using a small molecular PET radiotracer which has high affinity and selectivity to α-synuclein protein, the level of α-synuclein aggregation can be quantified. This approach not only improves the diagnostic accuracy of PD, but also provides a tool to monitor the progression of the disease and the efficacy of the treatment, and improve the understanding of disease progression.

Methods known in the art for radiolabeling the compounds of the present invention may be used. Reagents having a radionuclide that may be used in the preparation radiolabeled compounds of the present invention include for example [¹¹C]CH₃I.

The compounds of present invention may be formulated in a suitable pharmaceutical delivery medium or vehicle. In various embodiments, the pharmaceutical composition comprises an injectable comprising a compound of the present invention. In other embodiments, the pharmaceutical delivery medium comprises an oral vehicle comprising a compound of the present invention (e.g., capsule, pill, liquid, suspension, etc.).

### Synthesis

Representative synthetic methods are disclosed below. More specific synthesis conditions can be found in U.S. Patent Application Publication No. 2019/0256492, incorporated herein by reference for its synthetic methods.

All reagents and chemicals were purchased from commercial suppliers and used without further purification unless otherwise stated. Melting points were determined on a MEL-TEMP 3.0 apparatus and left uncorrected. ¹H NMR and ¹³C NMR spectra were recorded at 400 or 300 MHz on a Varian Mercury-VX spectrometer with CDCl₃, CD₃COCD₃, CD₃OD, CD₃SOCD₃ as solvent and tetramethylsilane (TMS) as the internal standard. All chemical shift values are reported in parts per million (ppm) (δ). With this condition, the chemical shifts (in ppm) of residual solvents are observed at 7.26 (CDCl₃), 2.05 (CD₃COCD₃), 4.80 (CD₃OD), 2.50 (CD₃SOCD₃) for ¹H NMR. The following abbreviations were used to describe peak patterns wherever appropriate: b=broad, d=doublet, t=triplet, q=quartet, m=multiplet. HRMS analyses were conducted in Washington University Resource for Biomedical and Bio-organic Mass Spectrometry. Preparative chromatography was performed on Chemglass chromatography column using 230-400 mesh silica gel purchase from Silicycle. Analytical TLC was carried out on Merck 60 F₂₅₄ silica gel glass plates, and visualization was aided by UV.

### Synthesis of Analogues Having Methoxy Groups on Quinolone Ring

New analogues possessing methoxy group on the 7- and 8-position of the quinoline ring were prepared with the goal of improving selectivity for α-synuclein versus Aβ and tau proteins. The palladium-catalyzed coupling reaction gave the product in higher yield than direct aromatic nucleophilic substitution. To confirm that the secondary NH plays an important role for the binding affinity, the secondary amine was converted to a tertiary amine with a methyl protection to give exemplary compound TZ61-66 (Scheme 1).

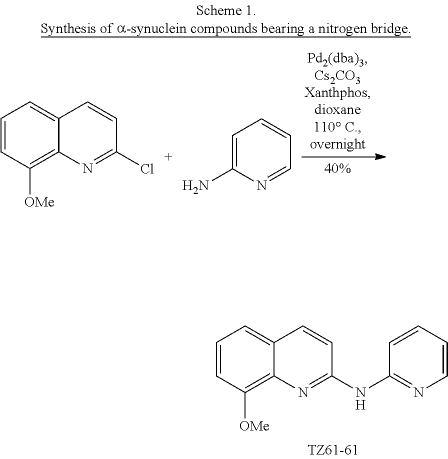 [SMILES: COc1cccc2ccc(Cl)nc12 ; Nc1ccccn1 ; COc1cccc2ccc(Nc3ccccn3)nc12]

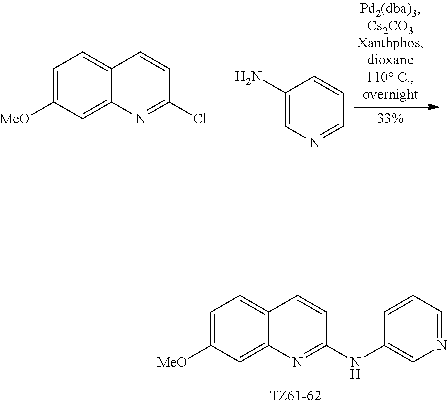 [SMILES: COc1ccc2ccc(Cl)nc2c1 ; Nc1cccnc1 ; COc1ccc2ccc(Nc3cccnc3)nc2c1]

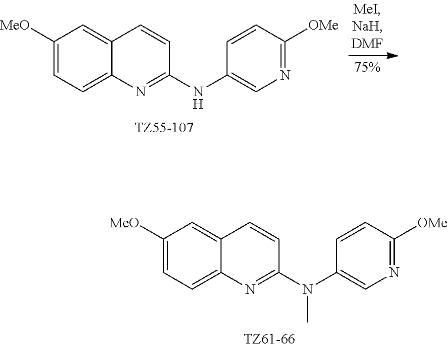 [SMILES: COc1ccc2nc(Nc3ccc(OC)nc3)ccc2c1 ; COc1ccc2nc(N(C)c3ccc(OC)nc3)ccc2c1]

### Production of [¹⁸F]Fluoride

[¹⁸F]Fluoride was produced by ¹⁸O(p, n)¹⁸F reaction through proton irradiation of enriched ¹⁸O water (95%) using a RDS111 cyclotron (Siemens/CTI Molecular Imaging, Knoxville, TN). [¹⁸F]Fluoride is first passed through an ion-exchange resin and then is eluted with 0.02 M potassium carbonate (K₂CO₃) solution.

### Synthesis of TZ61-23

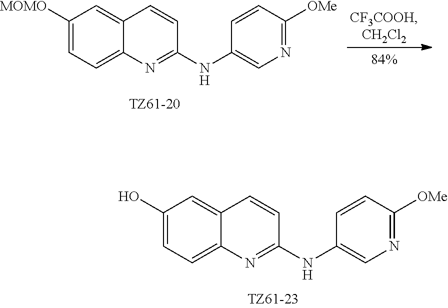

To a solution of MOMO group protected TZ61-20 (62 mg, 0.20 mmol) in anhydrous CH₂Cl₂ (2 mL) was added CF₃COOH (1 mL) at 0° C. The solution was stirred at room temperature overnight. Water was added to quench the reaction and the mixture was extracted with EtOAc, the combined organic phase was washed with saturated sodium bicarbonate, water, saturated sodium chloride, dried over anhydrous Na₂SO₄. The organic layer was concentrated and the residue was subjected to silica gel chromatography (hexane/EtOAc 1/1) to afford product TZ61-23 as a light yellow solid (45 mg, 84% yield).

### Synthesis of TZ61-44 (Non-Radiolabeled)

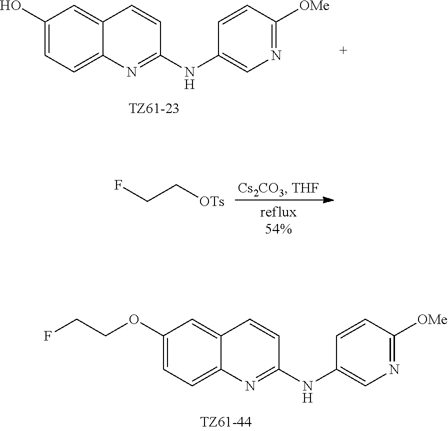 [SMILES: COc1ccc(Nc2ccc3cc(O)ccc3n2)cn1 ; Cc1ccc(S(=O)(=O)OCCF)cc1 ; COc1ccc(Nc2ccc3cc(OCCF)ccc3n2)cn1]

To a solution of TZ61-23, prepared as described in Example 3, (11 mg, 0.040 mmol) in anhydrous THF (2 mL) was added Cs₂CO₃ (21 mg, 0.064 mmol), 2-fluoroethyl tosylate (13 mg, 0.060 mmol) successively. The mixture was stirred at 70° C. in a sealed tube overnight. Water was added to quench the reaction and the mixture was extracted with EtOAc, the combined organic phase was washed with saturated sodium chloride, dried over anhydrous Na₂SO₄. The combined organic layer was concentrated and the residue was subjected to silica gel chromatography (hexane/EtOAc 1/1) to afford product TZ61-44 as a yellow solid (7 mg, 54% yield).

### A General Procedure for the Radiosynthesis of [¹⁸F]Fluoroethyl Tosylate

A sample of ˜200 mCi [¹⁸F]/fluoride was added to a reaction vessel containing K₂₂₂ (6-8 mg). The syringe was washed with 2×0.4 mL ethanol. The resulting solution was evaporated under nitrogen flow with a bath temperature of 110° C. To the mixture acetonitrile (3×1.0 mL) was added and water was azeotropically removed by evaporation. After all the water was removed, 5-6 mg of the corresponding precursor 1,2-ethylene ditosylate was dissolved in acetonitrile (200 μL) under vortex, and the precursor solution was transferred into the reaction vessel containing [¹⁸F]fluoride/K₂₂₂/K₂CO₃. The reaction tube was capped and the reaction mixture was briefly mixed, and then subjected to heating in an oil bath that was preheated to 110° C. for 10 min.

After heating for 10 minutes, the reaction mixture was diluted with 3.0 mL of HPLC mobile phase (50:50 Acetonitrile/0.1 M ammonium formate buffer (pH˜6.5)) and passed through an alumina Neutral Sep-Pak Plus cartridge. The crude product was then loaded onto an Agilent SB—C18 semi-preparative HPLC column (250 mm×10 mm) with a UV detector set at 254 nm. The HPLC system used a 5 mL injection loop. With 50:50 Acetonitrile/0.1 M ammonium formate buffer (pH˜6.5) as eluent at 4.0 mL/min flow rate, the retention time of the product was 10-11 min. The retention time of the precursor was 23-24 min. In situ monitored by the radioactivity detector, the HPLC collection was diluted with ˜50 mL sterile water and the diluted collection went through a C-18 Sep-Pak Plus cartridge to trap the [¹⁸F]fluoroethyl tosylate on the Sep-Pak. The trapped product was eluted with diethyl ether (3 mL).

The eluted solution formed two phases, the top ethereal phase was transferred out, and the bottom aqueous phase was extracted with another 1 mL of ether. The combined ether solution was passed through a set of two stacked Sep-Pak Plus dry cartridges into a reaction vessel. After the ether was evaporated with a nitrogen stream at 25° C. The dried tracer was used for next reaction.

General Procedure for the Conjugation of [¹⁸F]Fluoroethyl Tosylate with Precursor

1.0-1.5 mg (4-5 mg for ¹⁹F-MNI659) of precursor was dissolved in appropriate solvent and was transferred to a vial containing related base. After vortexing for 1 min, the solution was added into the reaction tube containing the activity. The tube was capped and briefly swirled with a vortex, and then was kept at appropriate temperature for 10 min or 15 min. Subsequently, the residual was diluted with 3 mL HPLC mobile phase and loaded onto a Semi-Prep HPLC system for purification. The HPLC system contains a 5 mL injection loop, a UV detector at 254 nm and a radioactivity detector, at 4.0 mL/min flow rate. After the HPLC collection was diluted with ˜50 mL sterile water, the product was trapped on a C-18 Sep-Pak Plus cartridge and washed with 20 mL water. The trapped product was eluted with ethanol (0.6 mL) followed by 5.4 mL of 0.9% saline. After sterile filtration into a glass vial, the final product was ready for quality control (QC) analysis and animal studies. An aliquot of sample was assayed by an analytical HPLC system. The sample was authenticated by co-injecting with the cold standard solution. The radiochemical purity was >99%. The entire procedure took ˜2 h for two-step radiolabeling and 3 hours for three-step radiolabeling.

Production of [¹¹C]CH₃I

Production of [¹¹C]CH₃I followed the reported method. Briefly, [¹¹C]CH₃I was produced on-site from [¹¹C]CO₂ using a GE PETtrace MeI Microlab. Up to 1.4 Ci of [¹¹C]carbon dioxide was produced from the JSW BC-16/8 cyclotron by irradiating a gas target of 0.5% O₂ in N2 for 15-30 min with a 40 μA beam of 16 MeV protons in the Barnard Cyclotron Facility of Washington University School of Medicine. After the GE PETtrace MeI Microlab system converted the [¹¹C]CO₂ to [¹¹C]CH₄ using a nickel catalyst [Shimalite-Ni (reduced), Shimadzu, Japan P.N.221-27719] in the presence of hydrogen gas at 360° C.; the [¹¹C]CH₄ was further converted to [¹¹C]CH₃I by reaction with iodine in the gas phase at 690° C. Approximately 12 min following the end-of-bombardment (EOB), several hundred millicuries of [¹¹C]CH₃I were delivered in the gas phase to the hot cell where the radiosynthesis was accomplished.

### A General Procedure for ¹¹C-Radiolabeling

[¹¹C]CH₃I was bubbled for a period of 2-3 min into a solution of precursor (1-2 mg) in appropriate solvent (0.25 mL) containing 3.0-5.0 μL of base solution (5.0 N) at room temperature. When the trap of radioactivity was completed, the sealed reaction vessel was heated at appropriate temperature for 5 min. After the heating source was removed, 1.75 mL of the HPLC mobile phase was added into to the reaction vessel. The mixture was loaded onto a reversed phase HPLC system to purify the mixture. Under these conditions, the product was collected into a vial that contained 50 mL aseptic water. After finishing the collection, the collected fraction was passed through a C-18 Plus Sep-Pak® cartridge to remove the mobile phase solvent, whereby target tracer was retained on the cartridge. Then the Sep-Pak cartridge was rinsed using 20 mL of sterile water. Finally, the tracer trapped on the Sep-Pak® was eluted with 0.6 mL of ethanol, following with 5.4 mL 0.9% sodium chloride solution, passing through a 0.22μ (Whatman Puradisc 13 mm syringe filter) sterile filter into a sterile pyrogen-free glass vial for delivery. For quality control, an aliquot of sample was assayed by an analytical HPLC system, UV at 254 nm. The sample was authenticated by co-injecting with the corresponding cold standard solution. The radiochemical purity was >99%. The radiosynthesis typically took 1 hour.

### Definitions

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include —OH. The “hydroxyl” can be optionally substituted.

The terms “halogen” and “halo”, as used herein, unless otherwise indicated, include a chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, include a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted. The amino group can preferably be —NR′R″, wherein R′ and R″ are independently hydrogen or an alkyl, substituted alkyl, aryl, substituted aryl, alkaryl, heterocyclo, or heteroaryl as defined herein.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C1-10 alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “carbonyl”, as used herein, unless otherwise indicated, can include a functional group consisting of a carbon atom double-bonded to an oxygen atom (C═O). The “carbonyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O-cycloheptyl, —O-cyclooctyl, —O-cyclononyl and —O-cyclodecyl, —O—CH2-cyclopropyl, —O—CH2-cyclobutyl, —O—CH2-cyclopentyl, —O—CH2-cyclohexyl, —O—CH2-cycloheptyl, —O—CH2-cyclooctyl, —O—CH2-cyclononyl, —O—CH2-cyclodecyl, —O—(CH2)2-cyclopropyl, —O—(CH2)2-cyclobutyl, —O—(CH2)2-cyclopentyl, —O—(CH2)2-cyclohexyl, —O—(CH2)2-cycloheptyl, —O—(CH2)2-cyclooctyl, —O—(CH2)2-cyclononyl, or —O—(CH2)2-cyclodecyl. An alkoxyl can be saturated, partially saturated, or unsaturated. The “alkoxyl” can be optionally substituted.

The term “cycloalkyl”, as used herein, unless otherwise indicated, can include an aromatic, a non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C3-10 cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include-lower alkyl-cycloalkyl, wherein lower alkyl and cycloalkyl are as defined herein. Examples of -lower alkyl-cycloalkyl groups include, but are not limited to, —CH2-cyclopropyl, —CH2-cyclobutyl, —CH2-cyclopentyl, —CH2-cyclopentadienyl, —CH2-cyclohexyl, —CH2-cycloheptyl, or —CH2-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S, and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “heterocyclic” can be optionally substituted.

The term “indole”, as used herein, is an aromatic heterocyclic organic compound with formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The term “substituted” can refer to replacing at least one hydrogen atom with at least one chemical group selected from the group consisting of hydroxyl, halo, alkyl, alkenyl, alkynyl, carboxyl, carbonyl, acetyl, cyano, amido, amino, heterocyclo, and aromatic ring.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such compound. Examples of solvates include compounds of the disclosure in combination with, for example, water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the disclosure. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or another counterion. The counterion may be any organic or inorganic moiety that stabilizes the charge on the parent compound. Furthermore, a pharmaceutically acceptable salt may have more than one charged atom in its structure. In instances where multiple charged atoms are part of the pharmaceutically acceptable salt, the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the disclosure. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the disclosure, or a salt thereof, that further can include a stoichiometric or non-stoichiometric amount of water bound by non-covalent intermolecular forces.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

It is to be understood that while the invention has been described in conjunction with the detailed description thereof, the preceding description is intended to illustrate and not limit the scope of the invention, which is defined by the scope of the appended claims. Other aspects, advantages, and modifications are within the scope of the following claims.

### Example 1: Radiosynthesis and Biological Evaluation of Two ¹⁸F-Labeled Pet Radioligands for α-Synuclein

The aggregation of misfolded α-synuclein protein is a pathological hallmark of Parkinson's disease (PD), Dementia with Lewy Bodies (DLB) and Multiple System Atrophy (MSA). In vivo imaging of α-synuclein aggregations with a clinically suitable positron emission tomography (PET) radioligand could provide important information in early diagnosis of PD, disease progression, and assessment of the efficacy of disease-modifying therapies. It is reported here the ¹⁸F radiolabeling and evaluation of two radioligands, [¹⁸F]TZ83-41 and [¹⁸F]TZ80-156, as potential imaging agents for α-synuclein aggregation.

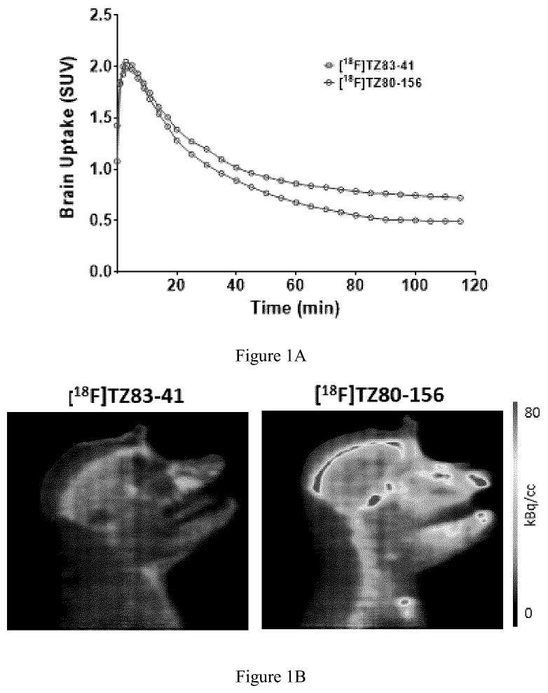

The radiosynthesis of [¹⁸F]TZ83-41 was accomplished by a two-step procedure with [¹⁸F]fluoroethyl ptoluenesulfonate as a prosthetic group, followed by O-alkylation to the quinolinyl-containing phenol group. [¹⁸F]TZ80-156 was synthesized starting from the allylic bromide precursor via nucleophilic halogen exchange (halex) reaction with [¹⁸F]fluoride, followed by deprotection of the N-Boc amine with trifluoroacetic acid. Homologous competitive binding assays with α-synuclein fibrils amplified from PD tissue were used to determine the direct binding affinity of the two radioligands. The biodistribution study was performed in male Sprague Dawley (SD) rats at four time points (5, 30, 60 min and 120 min) after intravenous injection of the radiotracer. The uptake of radioactivity was calculated as percentage injected dose per gram (% ID/gram). Both radiotracers were evaluated with microPET imaging using a Focus 220 microPET scanner to collect dynamic scans after intravenous injection into male macaques (NHP) (FIGS. 1A-1C).

The radioligands [¹⁸F]TZ83-41 and [¹⁸F]TZ80-156 were successfully produced in 17±3% (n=2) and 25±5% (n=2) radiochemical yield (RCY) respectively with high molar activity (>37 GBq/μmol) and high radiochemical purity (>99%), at the end of synthesis (EOS). Direct radioligand binding assays showed [¹⁸F]TZ83-41 and [¹⁸F]TZ80-156 have good to moderate binding affinity for α-synuclein fibrils (7.6 nM and 35 nM, respectively), as well as little to no binding to Alzheimer's disease brain tissue homogenate. MicroPET imaging revealed the brain standardized uptake value (SUV) for [¹⁸F]TZ83-41 and [¹⁸F]TZ80-156 reached a maximum (˜2.0) at 6-7 min post tracer injection, followed by a favorable washout rate from the NHP brain. Biodistribution studies indicated that [¹⁸F]TZ83-41 had a good brain uptake up to 0.71˜0.24% ID/gram from 5 to 120 min. For [¹⁸F]TZ80-156, the initial brain uptake (% ID/gram) was high with 0.86 at 5 min, but dramatically decreased to 0.25 at 30 min, and 0.16 at 60 min. Significant defluorination was observed in [¹⁸F]TZ80-156 since bone uptake was increased from 0.45 at 5 min to 2.18 at 30 min and 3.09 at 120 min.

Two ¹⁸F labeled radioligands, [¹⁸F]TZ83-41 and [¹⁸F]TZ80-156, were radiosynthesized and characterized. Both radiotracers crossed the blood brain barrier and possessed favorable washout kinetics in the NHP brain. Direct radioligand binding assays indicated higher binding affinity of [¹⁸F]TZ83-41 compared to [¹⁸F]TZ80-156 for α-synuclein fibrils. Defluorination of [¹⁸F]TZ80-156 makes [¹⁸F]TZ83-41 the better candidate for future translational investigations. Additional radiometabolism studies and characterization in corresponding disease models are warranted for clinical applications of this promising radiotracer.

### Example 2: NHP Plasma Radiometabolite Study Summary of α-Synuclein Radiotracer ([¹⁸F]TZ-83-41)

Monkey arterial/venous blood samples were collected at various intervals during the PET scans to measure the radio metabolite profile using the column-switching HPLC system equipped with an Oasis HLB online capture column (80 Å, 15 μm, 3.9 mm×20 mm, Waters 186001414) with Universal Guard Column holder (WAT046910, Waters) and a Agilent ZORBAX Eclipse XDB-C18 analytical column (5 μm, 250×4.6 mm, Part No. 990967-902), following a published protocol (Hilton J, et al., Nucl Med Biol. 2000 August; 27(6):627-30; Cai Z, et al., ACS Chem Neurosci. 2020 Feb. 19; 11(4):592-603).

Briefly, blood samples (vary with volume, 4-8 mL) were collected at 5, 15, 30, 60, 90 min post-injection of radiotracer. The whole blood was centrifuged at RCF 1300 g for 5 min to separate the blood cells and obtain plasma. Clean supernatant plasma was collected and added into solid urea to eliminate competitive protein binding and to allow complete backflush of the parent compound and metabolites from the capture column to analytical column. Typically, 3.2 mL of plasma is added to 2.12 g of urea to yield a final concentration of 8 M urea and a final volume of about 4.5 mL. The plasma-urea mix was then filtered through a Whatman® GD/X syringe filters (pore size 1 μm, diam. 13 mm, WHA68841310, Sigma, USA) and injected into a 5 mL HPLC loop concatenating with the capture column, which eluted with 1% acetonitrile in ammonium formate (0.1 M, pH=4.5) at 2 mL/min for 4 min. The enriched radioactivity in the short capture column was reverse flowed into an analytical C18 column with 60% acetonitrile and 40% ammonium formate (0.1 M, pH=4.5) at a flow rate of 1.0 mL/min for 25 min.

TABLE 1

| Time Point (min) | Peak A (%) | Peak B (%) |
|---|---|---|
| 5 | 7 | 92 |
| 15 | 31 | 69 |
| 30 | 41 | 59 |
| 60 | 53 | 47 |
| 90 | 62 | 38 |

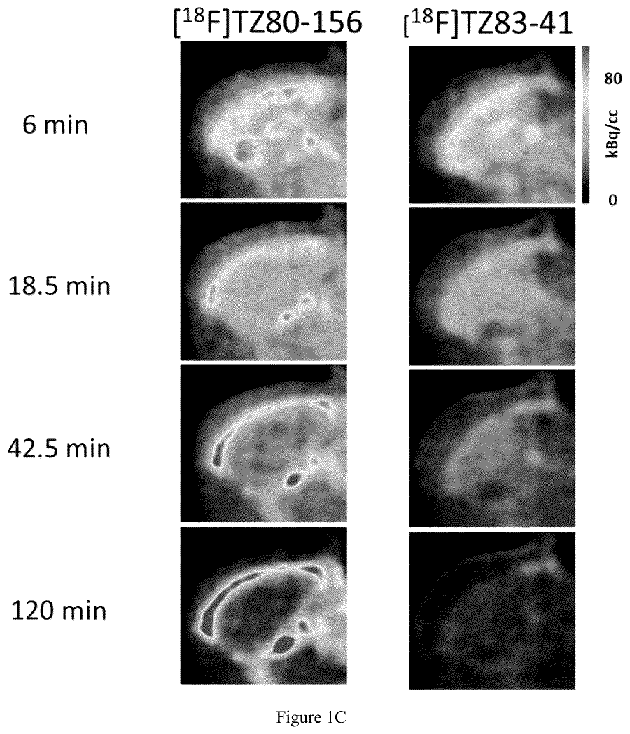

The radioactive peak B is the parental [¹⁸F]TZ83-41 tracer with retention time of 14.3 min; one radiometabolite is peak A at 6.8 min (FIG. 2A and FIG. 2B).

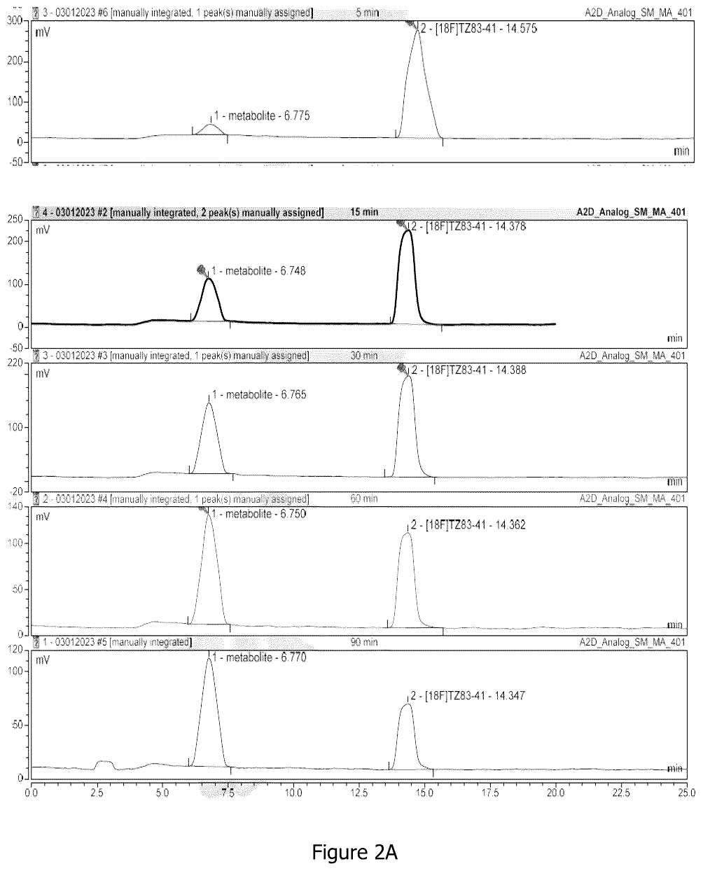

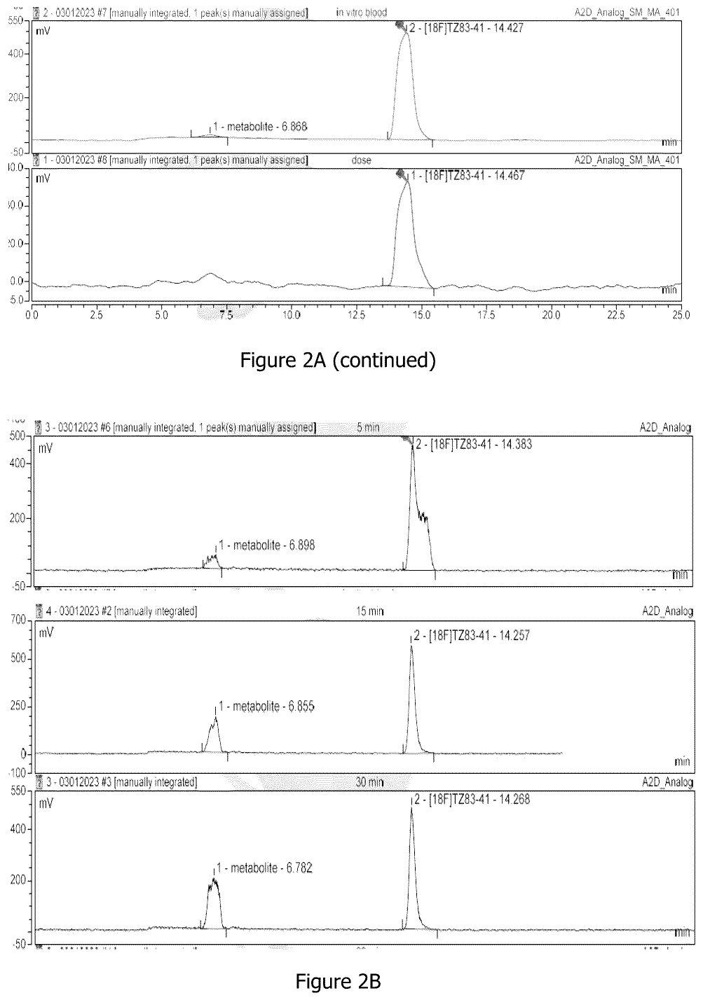

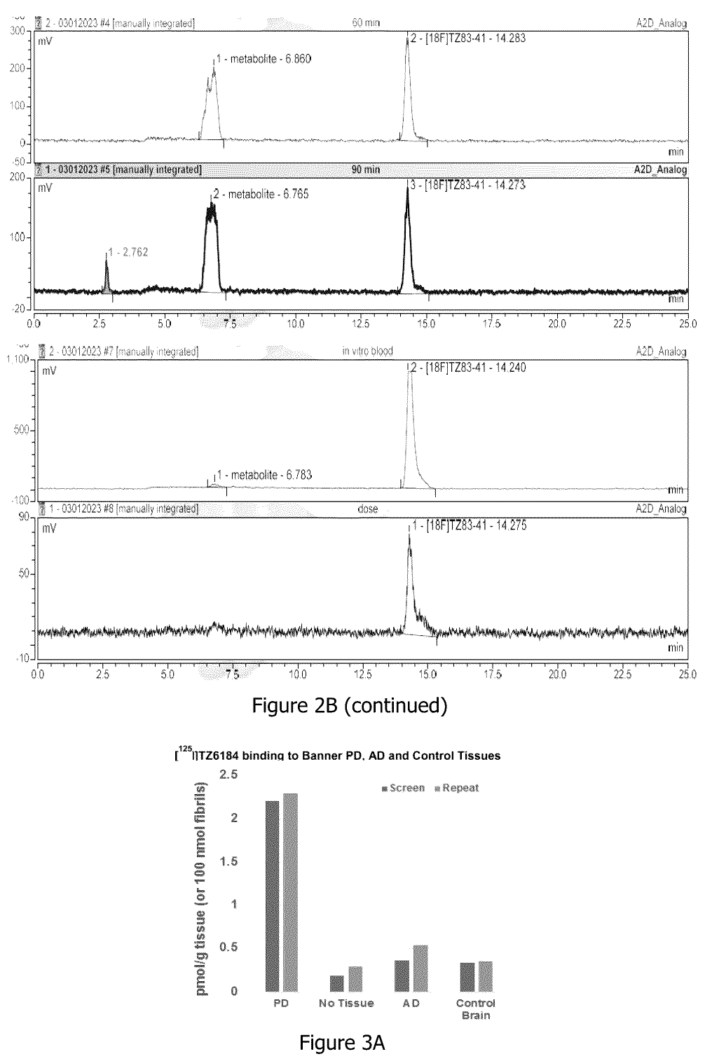

### Example 3: Pet Studies in the Brains of Nonhuman Primates

[¹¹C]TZ61-84 was shown to bind to Parkinson's disease brain tissue over control tissues (FIG. 3A). K_{d} and Bₘₐₓ values were determined for [¹²⁵I]TZ61-84 in this tissue, with K_{d} Hi=0.61 nM, Bₘₐₓ Hi=50 nM, K_{d} Lo=204 nM, and Bₘₐₓ Lo=12 μM (FIG. 3B). A PET study in nonhuman primates found uptake of [¹¹C]TZ61-84 (FIG. 3C).

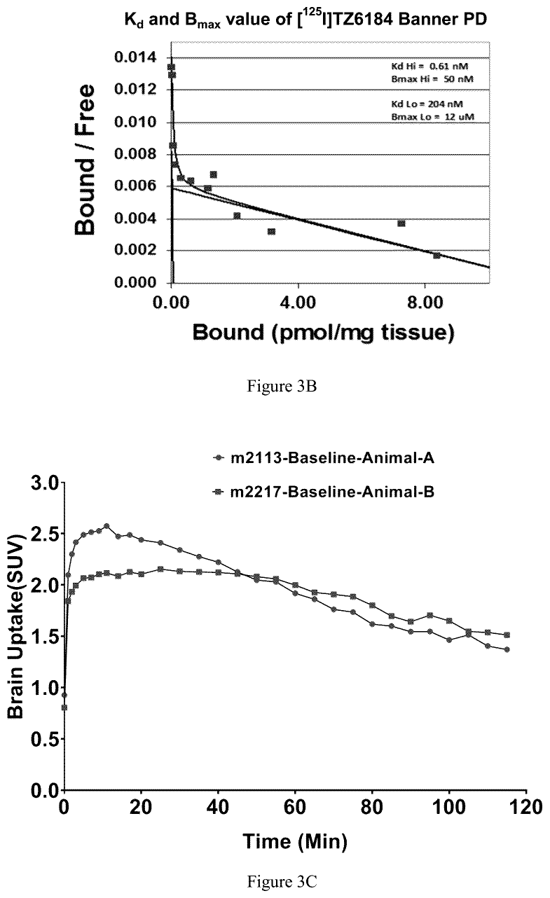

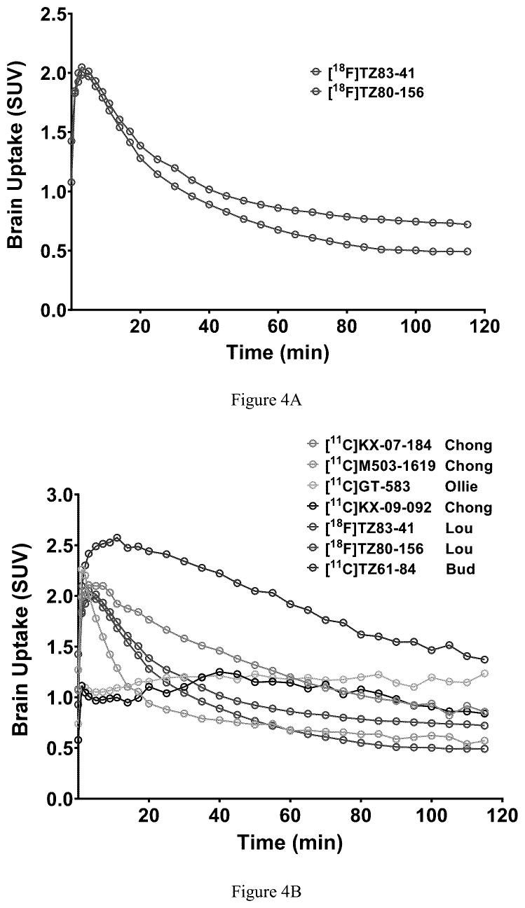

Another PET study of [¹⁸F]TZ80-156 and [¹⁸F]TZ83-41 in nonhuman primates also found uptake (FIG. 4A).

A further PET study also found uptake of several additional compounds, including [¹⁸F]TZ80-156 and [¹⁸F]TZ83-41 (FIG. 4B).

### Example 4: Binding Assay Summarys

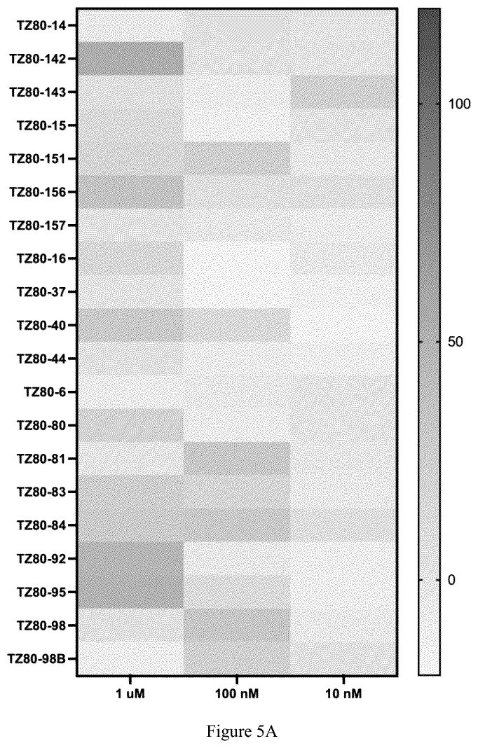

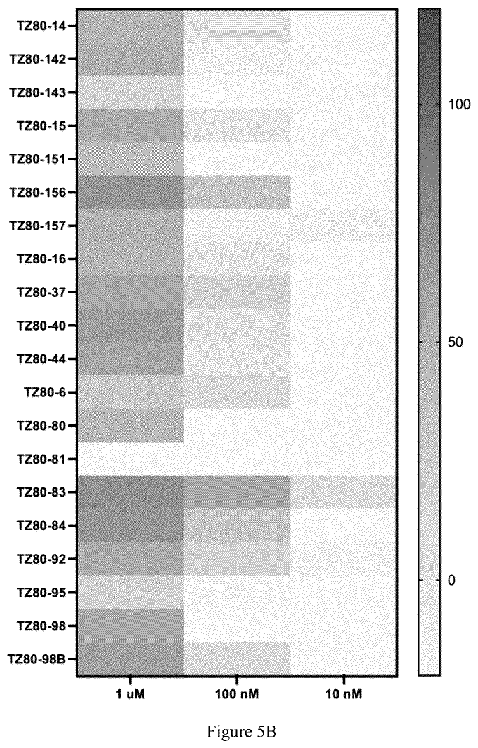

The alpha-synuclein fibrils binding potency of new compounds was determined by using triatiated [³H]BF2846, a reported potent and non-selective alpha-synuclein ligand (FIG. 5A). It showed most of these compounds have alpha-synuclein fibrils binding potency ranging from 100 nM-1 uM, ˜30 compounds have binding potency for alpha-synuclein fibril binding between 50-100 nM, and only a few compounds with binding potency between 10 to 50 nM.

The binding potency for Abeta was determined using [³H]PIB for these new compounds (FIG. 5B). [³H]PIB is a highly potent and selective a-beta radioligand. The data shows that most of these compounds also have good binding potency for a-beta brain tissue from AD patient brain. Most of these compounds have AD brain tissue binding ranging from 100 nM-1 uM, ˜20-30 compounds have binding potency for AD brain tissues between 50-100 nM, and only 3 compounds have binding potency for AD brain tissue with 10 to 50 nM.

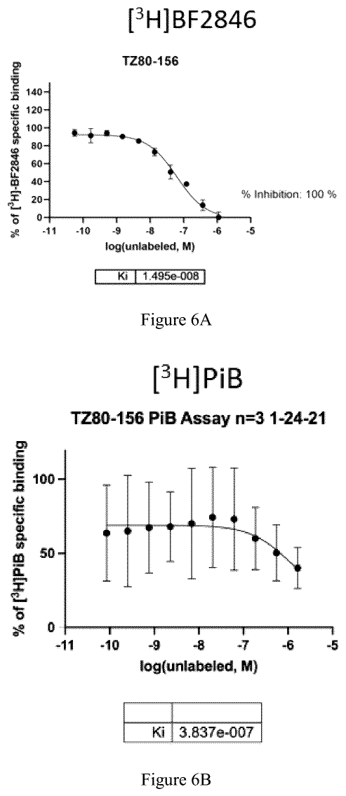

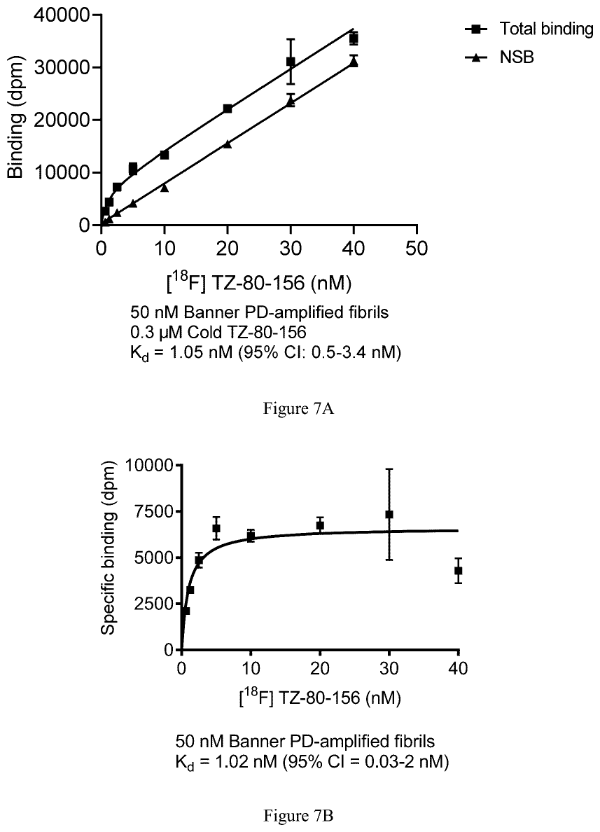

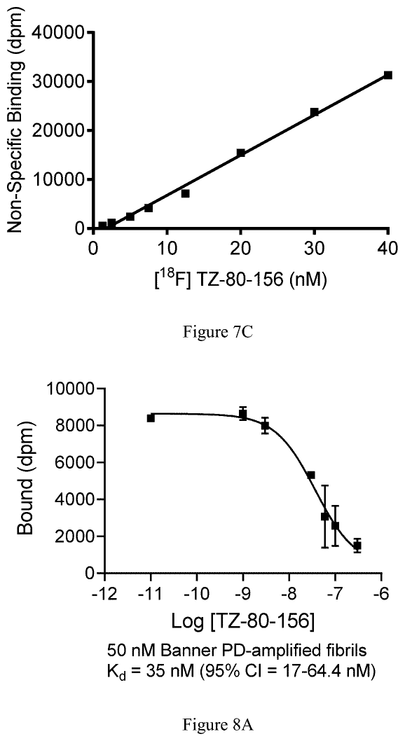

Specific binding for [3H]TZ80-156-related compounds was measured (FIGS. 6A-6B). Total binding, specific binding, and non-specification binding were also measured for [¹⁸F]TZ80-156 (FIGS. 7A-7C). [¹⁸F]TZ-80-156 was delivered at 2mCi in 200 uL (100% Ethanol) with a specific activity of 1611 Ci/mmol.

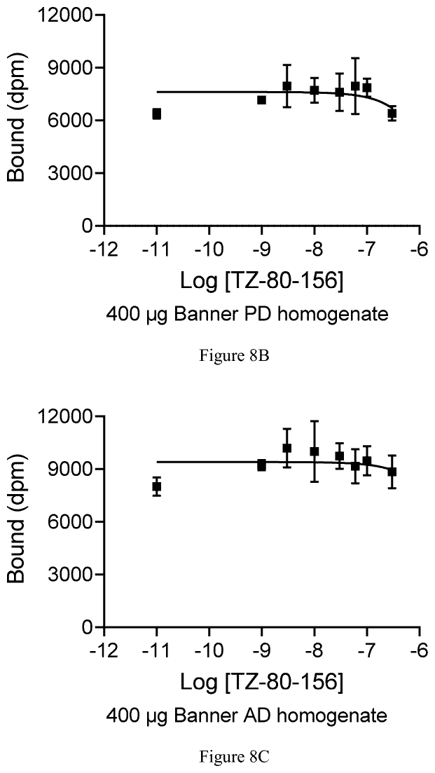

TZ80-156 binding to Banner PB-amplified fibrils (FIG. 8A), Banner PD homogenate (FIG. 8B), and Banner AD homogenate (FIG. 8C) were also measured via a homologous competition binding assay.

### Example 5: Exemplary Compounds

Exemplary compounds and their properties are shown in Tables 2-4. Table 2 shows introducing an amide or addition aromatic group significantly lowers the potency. Table 3 shows that most compounds have high binding for AD.

Ki means the binding potency for alpha-synuclein fibrils with these compound screened using [³H]BF2846, a potent alpha-synuclein radiotracer for binding site 9. The max % inhibition site 9 indicates how strong these compounds inhibit the [³H]BF2846 binding to alpha-synuclein fibrils for both specific binding and no specific binding.

Similarly, when using [³H]PIB screening for AD brain tissue binding, a large value of Ki>1000 indicates those compounds do not compete with [³H]PIB and these compounds have no binding with AD brain tissues. These compounds are more selective for alpha-synuclein if their Ki for alpha-synuclein fibrils is <50 nM. If the Ki value is smaller, these compounds are potent for AD brain tissue binding and are not selective for alpha-synuclein over abeta, or PD over AD.

TABLE 2

| | Ki (nM), | | | |
|---|---|---|---|---|
| | in a-syn | | | |
| | fibrils v.s. | max % | Ki (nM) in AD | max % |
| Name | [3H]BF2846 | inh_site9 | vs [3H]PiB | Inh_PiB |
| TZ80-106 | 15.04 ± 12.41 | 100 | >1000 | |
| TZ64-105 | 18.7 | 50 | >1000 | |
| TZ80-55 | 26.1 ± 93.79 | 70 | >1000 | |
| (HCl Salt) | | | | |
| TZ80-66-B | 30.4 ± 42.10 | 70 | 25.2 ± 21.7 | 80 |
| (HCl Salt) | | | | |
| TZ83-41 | 12.5 ± 40.81 | 60 | 362 ± 372 | 40 |
| (HCl Salt) | | | | |
| TZ80-156 | 14.95 ± 19.07 | 100 | >1000 | |
| TZ88-27 | None | | >1000 | |
| TZ80-30 | 14.3 ± 25.42 | 100 | 158 ± 36 | 80 |
| TZ80-86 | 31.3 ± 62.55 | 100 | 151 ± 103 | 70 |
| (HCl Salt) | | | | |
| TZ80-42 | 16.6 ± 22.64 | 70 | 72 ± 56 | 60 |
| TZ80-59-B | 17.9 ± 31.72 | 70 | 108 ± 73 | 100 |
| (HCl Salt) | | | | |
| TZ83-39 | 30.1 ± 304.33 | 60 | 142 ± 103 | 20 |
| (HCl Salt) | | | | |
| TZ88-11 | None | | 46 ± 78 | 70 |
| TZ80-37 | 22.9 ± 28.18 | 60 | 99 ± 90 | 70 |
| TZ64-91 | 1.4 | 70 | 53 ± 34 | 50 |
| TZ80-83 | 24.11 ± 39.67 | 100 | 50 ± 36 | 80 |
| TZ64-87 | 3.07 | 50 | 36 ± 30 | 30 |

TABLE 3

| | Ki (nM), | | | |
|---|---|---|---|---|
| | in a-syn | | | |
| | fibrils v.s. | max % | Ki (nM) in AD | max % |
| Name | [3H]BF2846 | inh_site9 | vs [3H]PiB | Inh_PiB |
| TZ61-84 | 21.5 ± 34.02 | 100 | 13.2 ± 17.2 | 90 |
| TZ80-64 | 17.9 ± 10.48 | 80 | >1000 | |
| (HCl Salt) | | | | |
| TZ88-5 | 55.58 ± 1042.87 | 40 | 7.1 ± 10.5 | 100 |
| TZ88-21 | None | | 5.8 ± 4.3 | 100 |
| TZ90-3 | 34.03 ± 75.29 | | 10.8 ± 8.3 | 100 |
| TZ90-4 | 46.22 ± 39.24 | Shallow | 3.1 ± 2.3 | 100 |
| | | Curve | | |
| TZ88-18 | None | | 1.6 ± 0.6 | 100 |
| TZ80-77 | 32.8 ± 43.80 | 100 | 15.0 ± 23.5 | 100 |
| (HCl Salt) | | | | |
| TZ88-7 | None | | 90 ± 155 | 90 |
| TZ64-73 | 4.6 | 20 | 6.6 ± 2.7 | 30 |
| TZ90-8 | 22.12 ± 25.37 | Shallow | 2.0 ± 2.6 | 100 |
| | | Curve | | |
| TZ83-40 | 13.3 ± 26.12 | 60 | 354 ± 407 | 40 |
| (HCl Salt) | | | | |
| TZ61-23 | 1.56 | 20 | 36 ± 37 | 30 |
| TZ90-21 | 129 ± 77.02 | | 0.19 | 30 |

TABLE 4

| | Ki (nM), | | | |
|---|---|---|---|---|
| | in a-syn | | | |
| | fibrils v.s. | max % | Ki (nM) in AD | max % |
| Name | [3H]BF2846 | inh_site9 | vs [3H]PiB | Inh_PiB |
| TZ64-91 | 1.4 | 70 | 53 ± 34 | 50 |
| TZ64-105 | 18.7 | 50 | >1000 | |
| TZ80-30 | 14.3 ± 25.42 | 100 | 158 ± 36 | 80 |
| TZ80-37 | 22.9 ± 28.18 | 60 | 99 ± 90 | 70 |
| TZ80-42 | 16.6 ± 22.64 | 70 | 72 ± 56 | 60 |
| TZ80-83 | 24.11 ± 39.67 | 100 | 50 ± 36 | 80 |
| TZ80-55 | 26.1 ± 93.79 | 70 | >1000 | |
| (HCl Salt) | | | | |
| TZ80-59-B | 17.9 ± 31.72 | 70 | 108 ± 73 | 100 |
| (HCl Salt) | | | | |
| TZ80-64 | 17.9 ± 10.48 | 80 | >1000 | |
| (HCl salt) | | | | |
| TZ80-66-B | 30.4 ± 42.10 | 70 | 25.2 ± 21.7 | 80 |
| (HCl Salt) | | | | |
| TZ80-86 | 31.3 ± 62.55 | 100 | 151 ± 103 | 70 |
| (HCl Salt) | | | | |
| TZ80-106 | 15.04 ± 12.41 | 100 | >1000 | |
| TZ80-156 | 14.95 ± 19.07 | 100 | >1000 | |
| TZ83-41 | 12.5 ± 40.81 | 60 | 362 ± 372 | 40 |
| (HCl Salt) | | | | |
| TZ61-84 | 21.5 ± 34.02 | 100 | 13.2 ± 17.2 | 90 |

The compound structures corresponding to the ones listed elsewhere in this application are shown below:

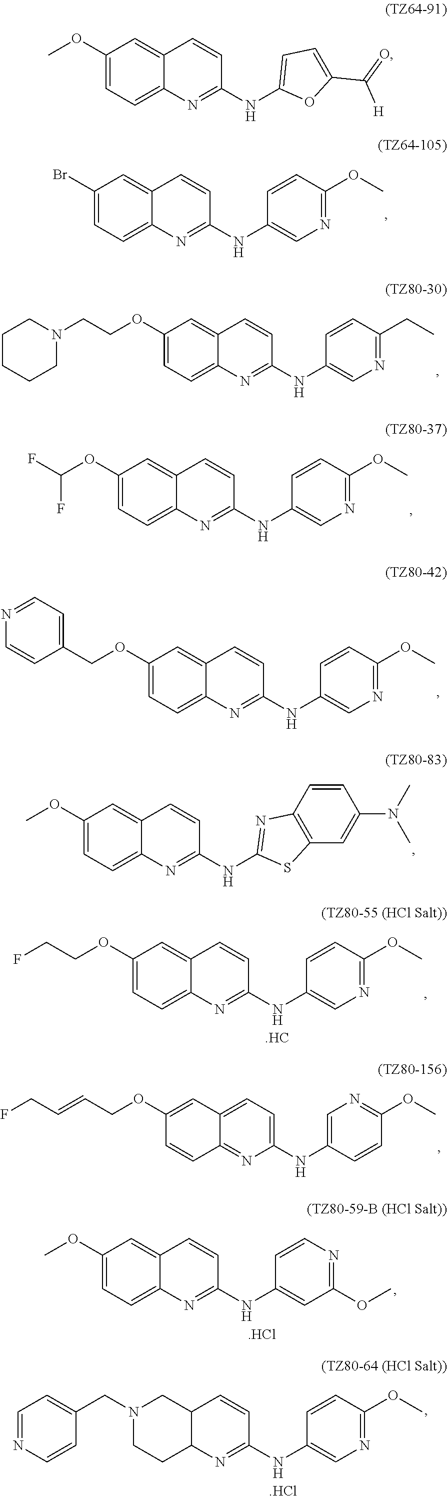

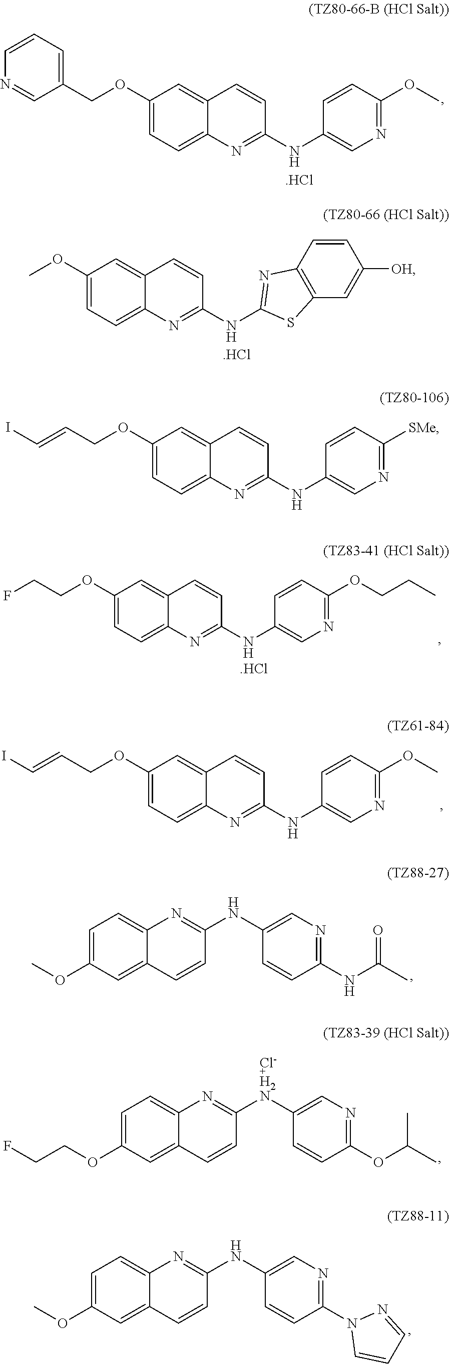

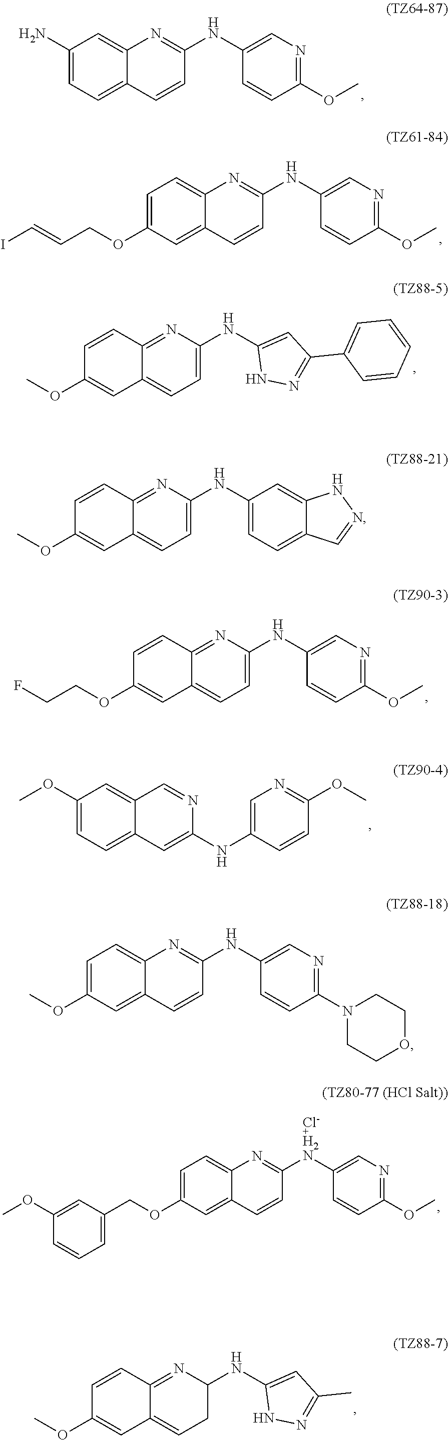

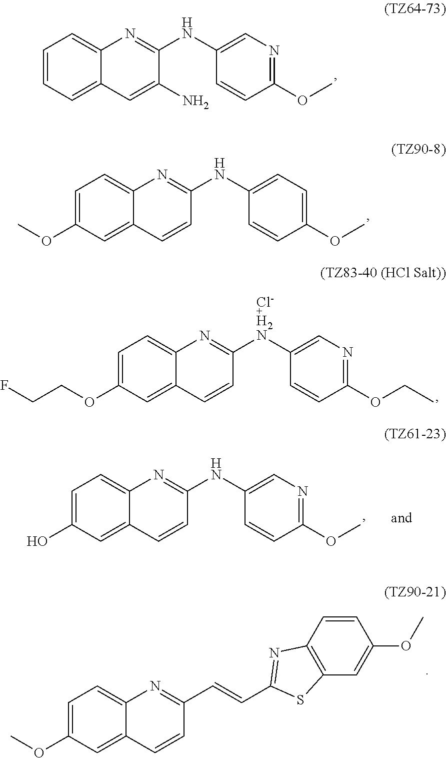

Exemplary radiolabeled compounds are shown below:

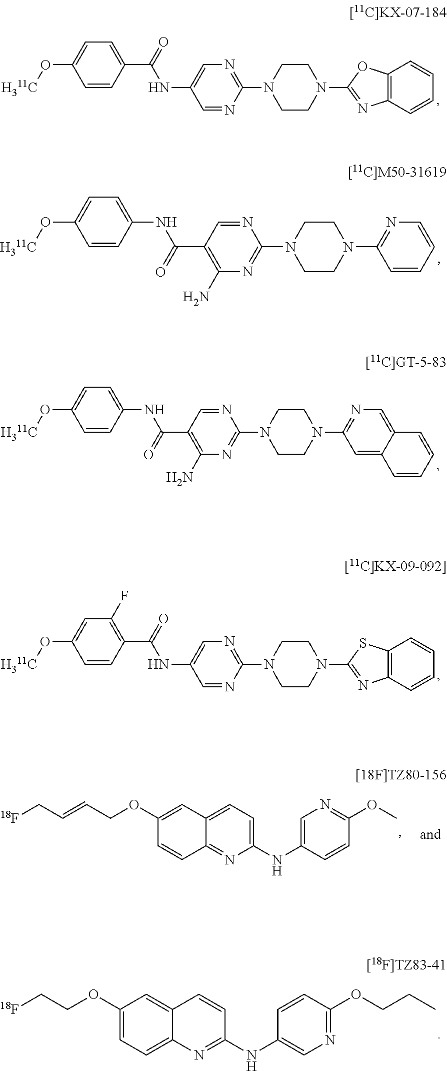 [SMILES: [11CH3]Oc1ccc(C(=O)Nc2cnc(N3CCN(c4nc5ccccc5o4)CC3)nc2)cc1 ; [11CH3]Oc1ccc(NC(=O)c2cnc(N3CCN(c4ccccn4)CC3)nc2N)cc1 ; [11CH3]Oc1ccc(NC(=O)c2cnc(N3CCN(c4cc5ccccc5cn4)CC3)nc2N)cc1 ; [11CH3]Oc1ccc(C(=O)Nc2cnc(N3CCN(c4nc5ccccc5s4)CC3)nc2)c(F)c1 ; COc1ccc(Nc2ccc3cc(OC/C=C/C[18F])ccc3n2)cn1 ; CCCOc1ccc(Nc2ccc3cc(OCC[18F])ccc3n2)cn1]

### Example 6: Exemplary Radiosynthesis of Compounds

Radiosynthesis of [¹⁸F]TZ83-41 is shown below. Properties of [¹⁸F]TZ83-41 when administered for a PET brain scan of a monkey are shown in Table 5.

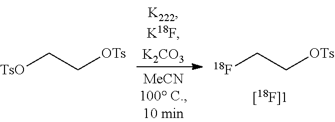 [SMILES: Cc1ccc(S(=O)(=O)OCCOS(=O)(=O)c2ccc(C)cc2)cc1 ; Cc1ccc(S(=O)(=O)OCC[18F])cc1]

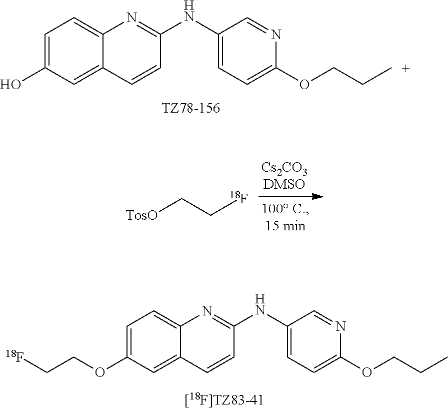

TABLE 5

| PET | | | | | |
|---|---|---|---|---|---|
| brain | Radioactivity | Radioactivity | Delivery | QC | SA |
| scan | [¹⁸F]1 | [¹⁸F]TZ83-41 | mCi | ppm | (Ci/mmol) |
| Monkey | 45 mCi | 12 mCi | 7 mCi | 0.42 | 1321@EOS |

Radiosynthesis of [¹⁸F]TZ80-156 is shown below, in addition to several unsuccessful synthesis examples.

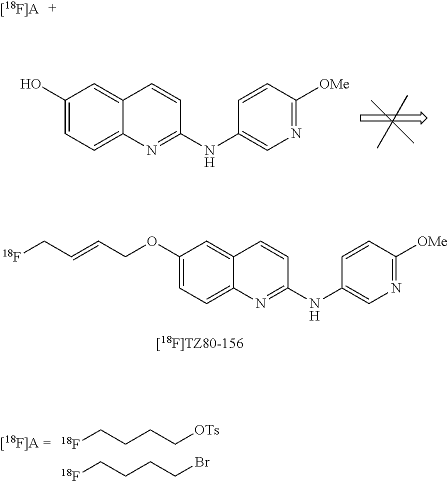

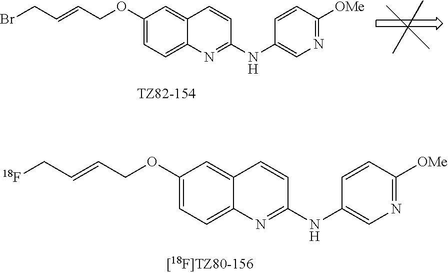 [SMILES: COc1ccc(Nc2ccc3cc(OC/C=C/CBr)ccc3n2)cn1 ; COc1ccc(Nc2ccc3cc(OC/C=C/C[18F])ccc3n2)cn1]

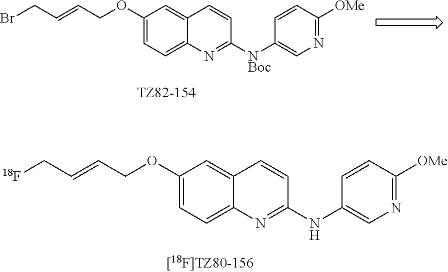 [SMILES: COc1ccc(N(C(=O)OC(C)(C)C)c2ccc3cc(OC/C=C/CBr)ccc3n2)cn1 ; COc1ccc(Nc2ccc3cc(OC/C=C/C[18F])ccc3n2)cn1]

Full radiosynthesis of [¹⁸F]TZ80-156 is shown below.

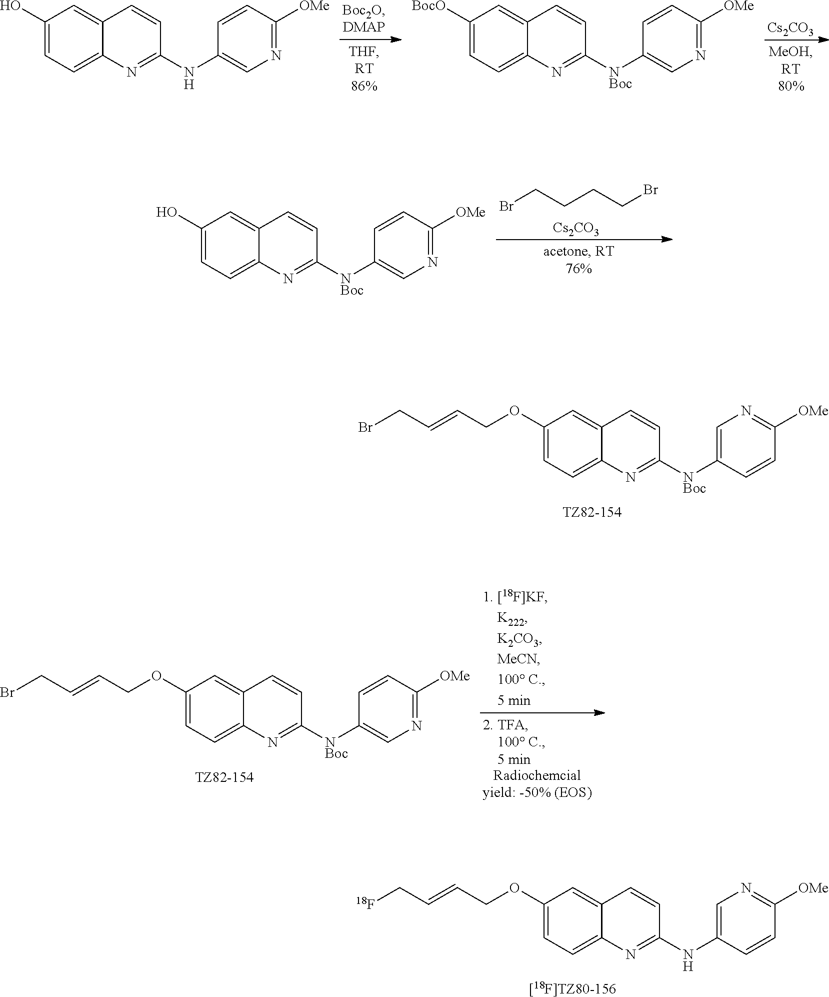

1. A compound having a structure of any one of Formulae 1, 2, 3, 4, or 5, or a salt thereof: 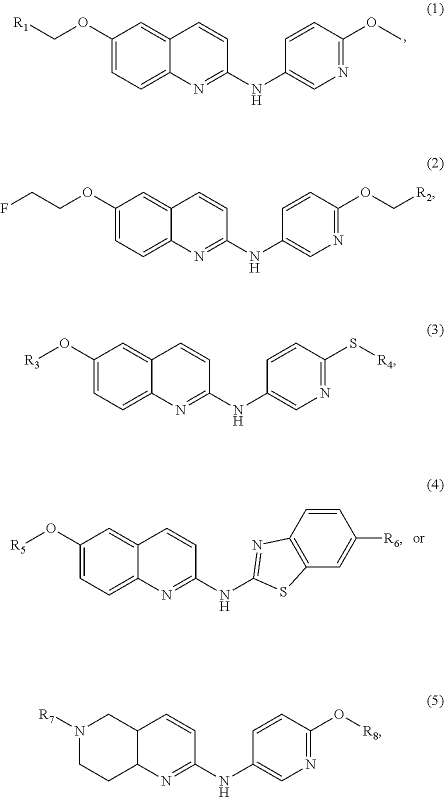
wherein:
R₁ is aryl, heteroaryl, halo-substituted C₃ alkyl, disubstituted halo-C₁-C₃ alkyl, trisubstituted halo-C₁-C₃ alkyl, cycloalkyl, alkylcycloalkyl, heterocycloalkyl, or alkylheterocycloalkyl;
R₂ is substituted C₁-C₅ alkyl or unsubstituted C₁-C₅ alkyl;
R₃, R₅, and R₈ are independently aryl, heteroaryl, alkaryl, alkheteroaryl, substituted C₁-C₆ alkyl, unsubstituted C₁-C₆ alkyl, substituted C₁-C₆ alkenyl, unsubstituted C₁-C₆ alkenyl, cycloalkyl, alkylcycloalkyl, heterocycloalkyl, or alkylheterocycloalkyl;
R₄ and R₆ are independently substituted C₁-C₆ alkyl, unsubstituted C₁-C₆ alkyl, substituted C₁-C₆ alkenyl, or unsubstituted C₁-C₆ alkenyl;
R₇ is hydrogen, C₁-C₆ alkyl, —OH, —OR₉, —NR₁₀R₁₁; and
R₉, R₁₀, and Ru are independently substituted C₁-C₆ alkyl or unsubstituted C₁-C₆ alkyl.

2. The compound of claim 1, having the structure of Formula 1, wherein R₁ is —CH₂-piperidyl, —CHF₂, —CF₃, pyridyl, or —CH═CH—CH₂F.

3. The compound of claim 1 having the structure of Formula 2A

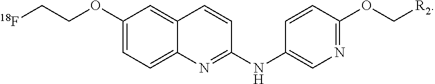

4. The compound of claim 3, wherein R₂ is methyl or ethyl.

5. The compound of claim 1 having the structure of Formula 3, wherein R₄ is C₁-C₃ alkyl.

6. The compound of claim 5, wherein R₄ is methyl.

7. The compound of claim 1 having the structure of Formula 4, wherein R₆ is —OH or —N(CH₃)₂.

8. The compound of claim 1, wherein R₅ is substituted C₁-C₃ alkyl or unsubstituted C₁-C₃ alkyl.

9. The compound of claim 8, wherein R₅ is halo-substituted C₁-C₃ alkyl.

10. The compound of claim 8, wherein R₅ is unsubstituted C₁-C₃ alkyl.

11. (canceled)

12. The compound of claim 1 having the structure of Formula 5, wherein R₈ is substituted C₁-C₃ alkyl or unsubstituted C₁-C₃ alkyl.

13. The compound of claim 1 having the structure of Formula 5, wherein R₈ is methyl.

14. The compound of claim 1, wherein R₇ is alkheteroaryl or —CH₂-pyridyl.

15. (canceled)

16. The compound of claim 1, wherein the structure is

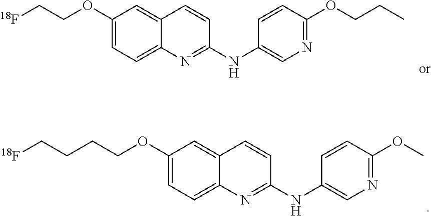 [SMILES: CCCOc1ccc(Nc2ccc3cc(OCC[18F])ccc3n2)cn1 ; COc1ccc(Nc2ccc3cc(OCCCC[18F])ccc3n2)cn1]

17. The compound of claim 1, wherein the compound comprises

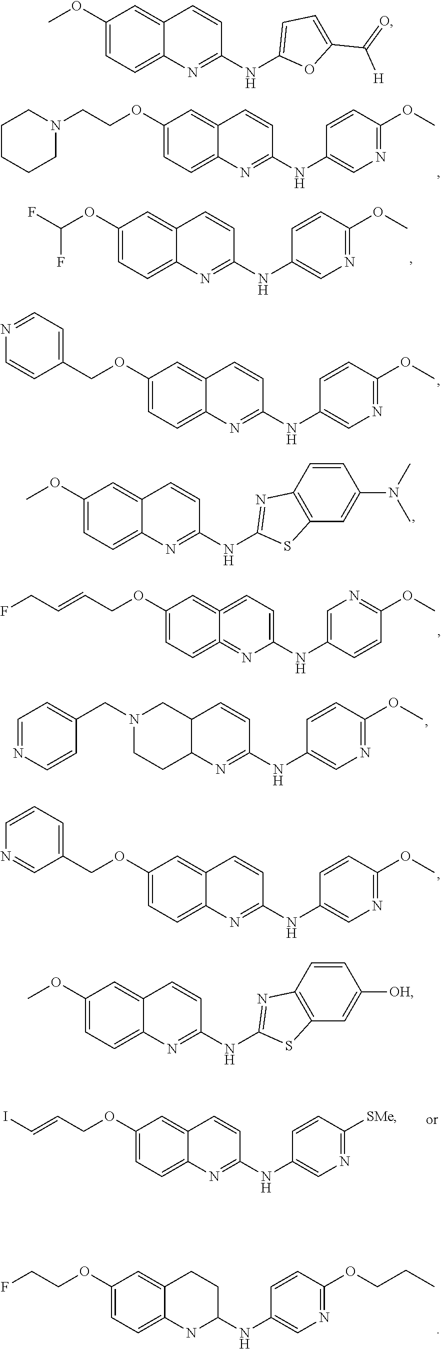

18. The compound of claim 1, wherein one or more atoms of the compound is radiolabeled with a synthetic radioactive isotope.

19. The compound of claim 18, wherein the synthetic radioactive isotope is carbon-11, nitrogen-13, oxygen-15, fluorine-18, bromine-76, iodine-123, or iodine-125.

20. The compound of claim 19, wherein the synthetic radioactive isotope is fluorine-18 or carbon-11.

21. A pharmaceutical composition comprising a radiolabeled compound of claim 1 and at least one excipient.

22. (canceled)

23. A method of diagnosing or monitoring a synucleinopathy in a human subject comprising administering the pharmaceutical composition of claim 21 to a human subject; and imaging the subject's brain by positron emission tomography.

24. (canceled)
